(19) United States
(12) Patent Application Publication
(10) Pub. No.: US 2025/0356249 A1
(43) Pub. Date: Nov. 20, 2025

(54) SYSTEM FOR SKIN TREATMENT VISUALIZATION AND PERSONALIZATION

(71) Applicant: ELC MANAGEMENT LLC, Melville, NY (US)

(72) Inventor: Christopher Aidan, Austin, TX (US)

(21) Appl. No.: 18/667,357

(22) Filed: May 17, 2024

(51) Int. Cl. G06N 20/00; G16H 50/20; G16H 50/70

(52) U.S. Cl. CPC G06N 20/00; G16H 50/20; G16H 50/70

(57) ABSTRACT

A system for providing a visualization of a skin care regimen, and techniques for generating a visualization of results of application of a skin care product or skin care regimen, are provided. Example methods may include obtaining training data including skin characteristics for a population of users, an indication of the respective skin care products used on the population of users, and a respective treatment outcome for each user. Methods may further include training a machine learning model, using the training data to predict a treatment outcome for a new user based on skin characteristics of the new user and on an indication of the skin care product used on the new user. Methods may further include providing a skin care regimen, based on the machine learning, for the new user. Methods may further include providing a visualization of the treatment outcome. Other systems, apparatuses and methods are described.

### FIELD OF THE INVENTION

The present invention relates generally to the field of personal care and, more specifically, to systems capable of providing visualization of skin care treatment effects using machine learning, artificial intelligence, augmented reality, and similar technologies.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The beauty and skin care industry provides a large array of products directed at changing the appearance of an individual's skin. However, the selection, effectiveness, and adherence to a schedule for using these products depends on individualized factors. Individuals may be more motivated to select and use some products if presented with visualizations of the effects of various products and treatments.

### SUMMARY

In one aspect, a computer-readable medium including instructions that, when executed on a processor, cause the processor to perform operations for providing a visualization of results of application of a skin care product. The operations can include obtaining training data including skin characteristics for a population of users, an indication of the respective skin care products used on the population of users, and a respective treatment outcome for each user; training a machine learning model, using the training data, to predict a treatment outcome for a new user based on skin characteristics of the new user and on an indication of the skin care product used on the new user; generating a visualization of the treatment outcome based on applying the trained machine learning model to an image of the new user; and providing the visualization to a display.

In another aspect, a method of providing a skin care augmented reality visualization can include accessing an image of a user; analyzing skin characteristics of the user based on the image; providing a skin care regimen based on the skin characteristics; generating a visualization of the image as the image would appear, after a time lapse, with implementation of the skin care regimen; and providing the visualization to a display.

In yet another aspect, a system for providing a skin care augmented reality visualization can include an image system configured to provide an image of a user; a display for displaying the image; and one or more processors coupled to the image system and to the display, the one or more processors configured to: analyze skin characteristics of the user based on the image: train a machine learning model to generate a skin care regimen based on the skin characteristics and on product information for skin care products; generate a visualization of the image as the image would appear, after a time lapse, with implementation of the skin care regimen; and provide the visualization to the display.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof.

There are shown in the drawings arrangements which are presently discussed, it being understood, however, that the present embodiments are not limited to the precise arrangements and instrumentalities shown, wherein:

FIG. 1 depicts an exemplary computer system for providing a visualization of results of a skin care regimen or application of a skin care product, according to some embodiments;

FIG. 2 depicts a three-dimensional face model creation process according to some embodiments;

FIGS. 3A-3C depict a user interface according to some embodiments;

FIG. 4 depicts an example visualization that may be provided by a user interface associated with a system according to some embodiments; and

FIG. 5 depicts a flow diagram of an exemplary computer-implemented method for providing a visualization of effects of a skin care regimen, according to some embodiments.

While the systems and methods disclosed herein are susceptible of being embodied in many different forms, they are shown in the drawings and are described herein in detail specific exemplary embodiments thereof, with the understanding that the present disclosure is to be considered as an exemplification of the principles of the systems and methods disclosed herein and is not intended to limit the systems and methods disclosed herein to the specific embodiments illustrated. In this respect, before explaining at least one embodiment consistent with the present systems and methods disclosed herein in detail, it is to be understood that the systems and methods disclosed herein are not limited in its application to the details of construction and to the arrangements of components set forth above and below, illustrated in the drawings, or as described in the examples.

Methods and apparatuses consistent with the systems and methods disclosed herein are capable of other embodiments and of being practiced and carried out in various ways. Also, it is to be understood that the phraseology and terminology employed herein, as well as the abstract included below, are for the purposes of description and should not be regarded as limiting.

### DETAILED DESCRIPTION

### Overview

The present disclosure provides systems for helping an individual visualize near and long-term future effects of following or not following a skin care regimen, and of applying any individual skin care product. Aging is an inevitable process that entails various changes in an individual's appearance, particularly in the skin. A wide range of products and treatments are available counteract these changes, or to treat or counteract other skin conditions such as acne, oiliness/dryness, and the like. However, the selection and effectiveness of products, and the likelihood that a user will adhere to a skin care regimen, depend greatly on individualized factors such as skin type, age, genetic predisposition, lifestyle, and specific aging patterns. Traditional methods of recommending treatments often lack personalization and fail to provide a clear visualization of potential outcomes. Moreover, there is a general lack of tools that can accurately show the possible consequences of non-adherence to these treatments.

Systems and methods according to aspects of this disclosure may address these and other concerns by generating a visualization of the potential effects of various products. Visualization may be provided using a display, e.g., virtual reality (VR) or augmented reality (AR) displays, and the like. The system can also generate visualization of the user's potential skin aging process in the absence of any treatments, enabling users to understand the possible outcomes of non-adherence to the recommended treatments.

The system described herein may employ machine learning algorithms to help enhance the accuracy of treatment-outcome matching and to help refine the visualization of the user's potential skin condition under different treatment scenarios. The system described herein may train artificial intelligence (AI) models to personalize recommended regimens based on skin characteristics, historical skin data, lifestyle, genetic factors, and user interaction data.

### Example System

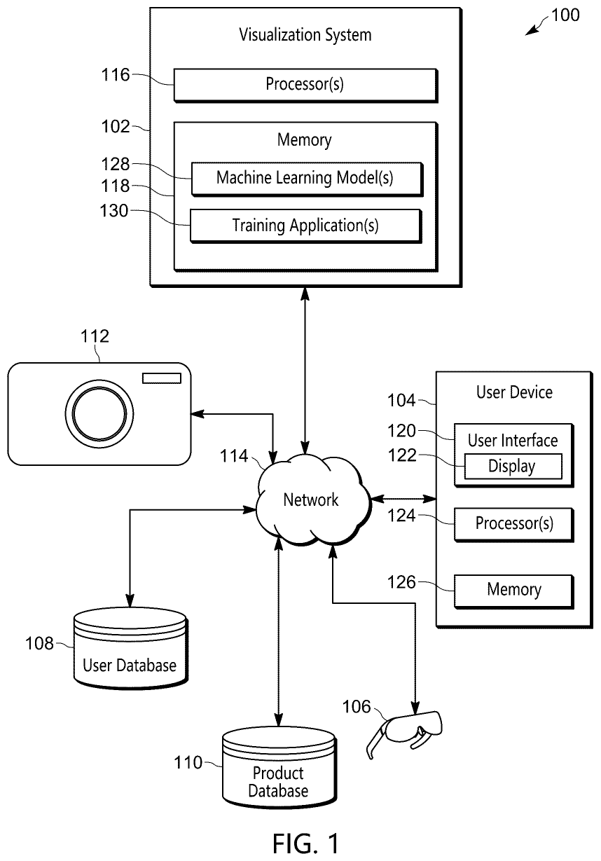

FIG. 1 depicts an exemplary computer system 100 for providing visualization of a skin care regimen or application of a skin care product, according to one embodiment. The high-level architecture illustrated in FIG. 1 may include both hardware and software applications, as well as various data communications channels for communicating data between the various hardware and software components, as is described below.

The system 100 may include a visualization system 102 as well as, in some cases, one or more user computing devices 104 (which may include, e.g., smart phones, smart watches or fitness tracker devices, tablets, laptops), and one or more display device(s) e.g., virtual reality headsets, smart or augmented reality glasses, wearables, etc.), 106. Data can be stored in separate databases either remotely or locally relative to the visualization system. For example, a user database 108 can include demographic data, medical data, genetic data, etc. of a user, and a product database 110 can include product names, formulations, and the like. The system 100 can include an imaging system 112 (e.g., a camera), which can be included in one or a plurality of locations in the system, for example, within the visualization system 102, user device 104, or as a separate standalone device. The visualization system 102, user device(s) 104, display device(s) 106 and/or imaging system 112 may be operable to communicate with one another via a wired or wireless computer network 114, and/or via short range signals, such as BLUETOOTH signals. In some example embodiments, some components or subsets of components of the visualization system 102 can be included within user device(s) 104 or display device(s) 106. For example, the imaging system 112 can include or comprise a camera of the user device 104, the display device 106 can include other components of a user device 104 (e.g., processor and memory, user interface components, and the like).

Although one visualization system 102, one user device 104, one display device 106, one imaging system 112 and one network 114 are shown in FIG. 1, any number of such visualization systems 102, user devices 104, display devices 106, imaging systems 112 and networks 114 may be included in various embodiments. To facilitate such communications, the visualization system 102, user devices 104, display devices 106 and/or imaging systems 112 may each respectively comprise a wireless transceiver to receive and transmit wireless communications.

The imaging system 112 can capture image(s) of the user's skin at one or more points in time so that the visualization system 102 (or components thereof) can perform time-based analysis of the effectiveness of products, changes due to time of year, and the like. As described later herein, components of the system 100 can use images, measurements, etc. in machine learning algorithms or other processing to perform predictions, provide product recommendations, and the like. The visualization system 102 can control the imaging system 112 to capture periodic images or on-demand images based on requests from the visualization system 102, the user device 104, the display device 106 or any combination or subset thereof.

The user device 104 includes a user interface 120 operable to receive inputs and selections from the user of the system 100 (e.g., the end user or customer), and/or to provide audible or visual feedback to the user.

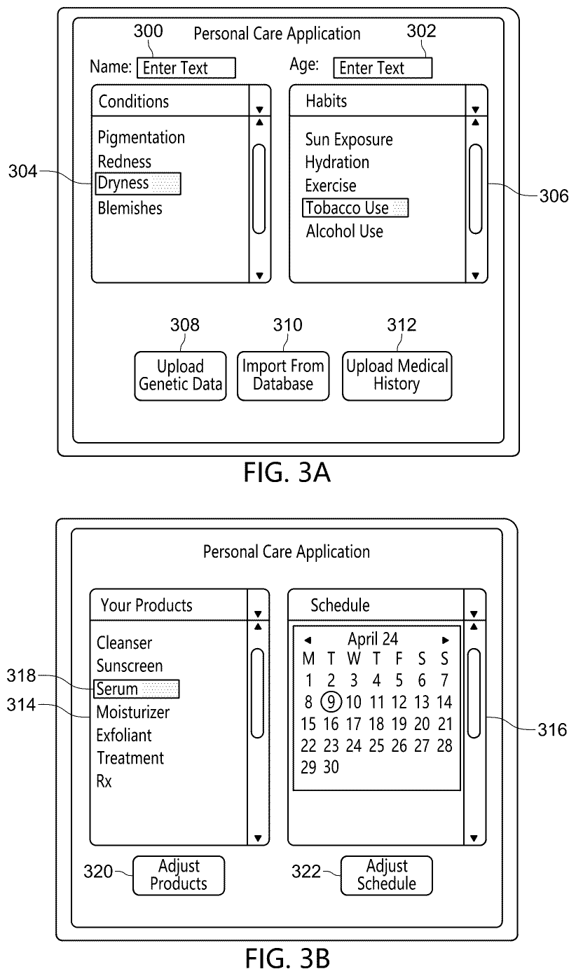

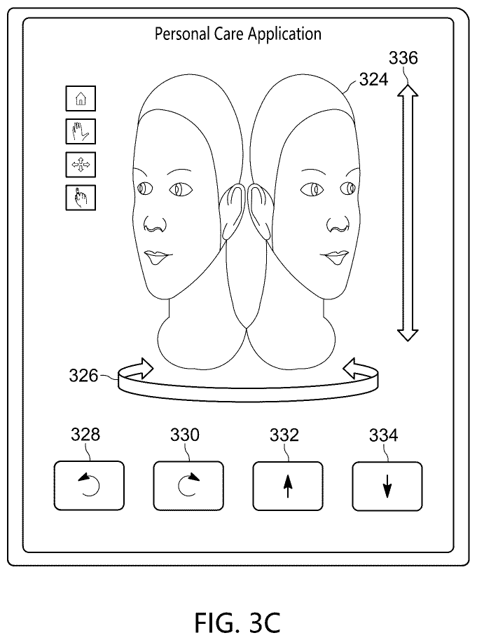

For instance, the user interface 120 may provide interactive displays via which users allows the user to interact with the system as described later herein with respect to FIGS. 3A-3C. For example, the user interface 120 can allow the user to input demographic information, lifestyle habits, medical history, and genetic data. The user interface 120 can include fields for entering data, uploading files, and importing data from external databases, among other functionalities and features.

In some examples, the user interface 120 may further include a display 122. The display 122 can include an augmented reality (AR) component operable to generate and display an AR rendering of a three-dimensional (3D) map of the user's face. In some cases, the AR rendering may be overlaid upon an image or video of the user's face as captured in real-time by the imaging system 112. The AR technology can also be used to provide users with a visual simulation of potential future skin conditions based on their personalized beauty regimen. The AR technology can additionally or alternatively be provided in a separate display device 106.

In some examples, the user interface 120 may be provided wholly or partially on a wearable device or an Internet of Things (IoT) device. Health data can be collected wholly or partially from the wearable device or IoT device.

Moreover, in some examples, the user interface 120 may be operable to receive feedback from a user. For example, a user, group of users or type of users may provide feedback on the perceived accuracy of the visualization, accuracy of predictions, results of recommended skin care recommendations, satisfaction with the visualization or other aspects of the skin care regimen and the like. The feedback can be provided to machine learning algorithms to improve predictions, product recommendations, regimen recommendations and the like by analyzing patterns in user feedback and to visualization software/systems to improve visualizations. Feedback can include automated or user-independent feedback capture including analyzing text reviews for sentiment, categorizing feedback into different themes, and identifying common issues or praises.

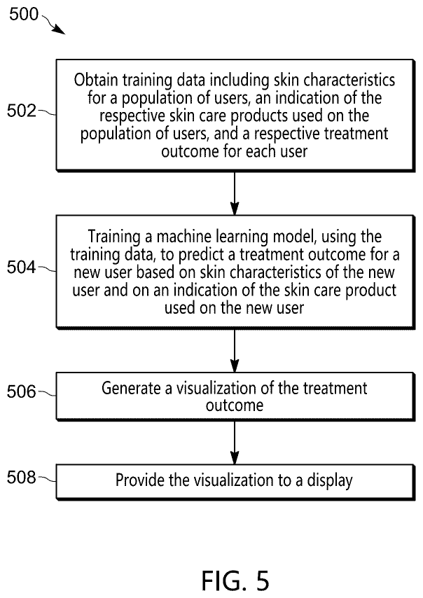

Moreover, the user device 104 may include one or more processor(s) 124, as well as one or more computer memories 126. Memories 126 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memories 126 may store an operating system (OS) (e.g., iOS, Microsoft Windows, Linux, UNIX, etc.) capable of facilitating the functionalities, apps, methods, or other software as discussed herein. The memories 126 may store instructions that, when executed by the processor(s) 124, cause the processor(s) 124 to receive input from a user as provided via the user interface 120 and send the received user input to the visualization system 102 (e.g., via the network 114) and/or to the imaging system 112 (when separate from the user device 104) and/or to the display device 106 (when separate from the user device 104), in some cases responsive to a request for such user input from the visualization system 102, the imaging system 112, and/or the display device 106. Furthermore, in some examples, the instructions stored on the memories 126 may cause the processor(s) 124 to perform any or all of the steps of the method 500 discussed below with respect to FIG. 5.

The visualization system 102 is configured to access images of a user. The images can include still photographic images, photographic video images, thermal image data, LiDAR or other laser-based image data, and/or other image data suitable for generating visualizations or other technologies of this disclosure. In some examples, the images can be produced or obtained from the imaging system 112.

The visualization system 102 can analyze skin characteristics of the user based on the image and the visualization system 102 can generate a skin care regimen based on the skin characteristics. Characteristics can include evidence of sun damage (such as wrinkling, hyperpigmentation, loss of skin tone, change in skin texture, and the like), any signs of acne (e.g., pimples, blackheads, whiteheads and the like), allergic reactions, eczema, general dryness, and the like. The visualization system 102 can generate a three-dimension (3D) representation of the user's face, as will be described in more detail later herein. The visualization system 102 can use images captured at different points in time to detect changes in oil and moisture saturation of the user's skin, reactivity of the user's skin to a specific substance, changes in visual evidence of sun damage, acne and the like, or any other condition. Any or all of the above visualization system 102 functions can additionally or alternatively be performed in other components of the system 100 (e.g., the user device 104, the display device 106, or any other device not shown connectable through the network 114).

The visualization system 102 can include one or more processor(s) 116, as well as one or more computer memories 118. The memories 118 may include one or more forms of volatile and/or non-volatile, non-transitory, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memories 118 may store an operating system (OS) (e.g., iOS, Microsoft Windows, Linux, UNIX, etc.) capable of facilitating the functionalities, apps, methods, or other software as discussed herein.

Generally speaking, the memories 118 may store instructions that, when executed by the processor(s) 116, cause the processors 116 to receive images from the imaging system 112. The memories 118 can cause the visualization system 102 to control image capture schedules and the like and to encode messages for communicate to the network 114.

Additionally, or alternatively, the memories 118 may store product data, including product identifiers and ingredients, which can be updated by product manufacturers in real-time. Product data may also be stored in a product database 110 (or in multiple such databases), which may be accessible or otherwise communicatively coupled to the visualization system 102.

The memories 118 may store user data. The user data may include previous products used by the user, user preferences, and various other data associated with the user, and may also be stored in a user database 108 (or in multiple such databases), which may be accessible or otherwise communicatively database coupled to the visualization system 102. Furthermore, in some examples, the product data and the user data may be stored in the same database, which may be accessible or otherwise communicatively coupled to the visualization system 102.

Furthermore, the memories 118 may store instructions that, when executed by the processors 116, cause the processors 116 to receive data from various databases such as the user database 108 and the product database 110, and/or data from the imaging system 112 and/or the user device 104 (e.g., via the network 114). The data from the imaging system 112 and/or the user device 104 may include, for instance, images, data input by a user via a user interface 120 of the user device 104, etc. The instructions stored on the memories 118, when executed by the processors 116, may cause the processors 116 to analyze data received from the database, and/or the imaging system 112 and/or the user device 104 to make a recommendation or prediction based on the received data, and subsequently send the recommendation and/or prediction to the user device 104.

The instructions stored on the memories 118 can further cause the processors 116 to generate updates to visualizations as described later herein. Furthermore, in some examples, the instructions stored on the memories 118 may cause the processor(s) 116 to perform any or all of the steps of the method 500 discussed below with respect to FIG. 5.

The memories 118 may store one or more machine learning models 128, and/or one or more respective machine learning model training applications 130 and the processor(s) 116 can execute or implement machine learning models 128 and machine learning model training applications 130. These machine learning models 128 may include, for instance, a machine learning model trained to analyze genetic data, imaging system 112 data (e.g., images, video, stills, etc.), lifestyle factors, social media inputs, geographical information, and other relevant input data to generate a personal care (e.g., skin care or beauty care) regimen for a user of the system 100. Example regimens can include lists of products or groups of products. For example, a recommendation could direct a user to include an exfoliant or moisturizer in the user's skin care regimen, to use a cleanser formulated for dry skin rather than oily skin, etc. In some embodiments, schedules can be provided. For example, a user may be directed to use some types of exfoliants only once per week, and at night rather than in the morning.

The machine learning model and/or other software applications or modules can refine visualizations of the user's potential skin condition under various treatment scenarios and can update visualizations or provide user feedback to refine the machine learning models themselves. As such, by implementing or executing the machine learning models 130 or other software applications/modules, the processor 116 can generate a visualization of treatment outcomes on a user. The processor 116 can obtain data including formulation information for products in the skin care regimen (e.g., from product database 110). Based on the effect these products or ingredients thereof had on a population similar to the user (e.g., a population of users similar in genetics, geographic location, or other characteristic or variable known or likely to affect reaction to products or medications), machine learning models 130 or other types of software applications/modules can predict the effect of that product on a particular user, and a visualization can be provided that takes into account that effect. For example, user using a particular moisturizer may be provided with a visualization of changes brought about by the moisturizer's use. Example changes that could be visualized may include changes common to persons of similar genetics, e.g., hyperpigmentation, tendency for reduced elasticity or wrinkling, acne, and the like.

The visualization system 102 can use the machine learning models 128 or other software program or module to track and analyze the impact of seasonal changes on skin health, taking into consideration factors such as humidity, temperature, and sunlight exposure. The machine learning models 128 adjust the personalized beauty regimen accordingly to optimize skin health in different seasons or other software programs/modules can determine or retrieve expected correlations of skin care conditions to these or similar seasonal changes. The machine learning models 128 can be trained to provide predicted outputs based on the influence of geographical location and local environmental factors on skin health. The visualization can be updated based on geographical location and local environmental factors by, e.g., changing skin tone of a visualization based on time of year or known sun, wind, or cold exposure. The machine learning models 128 can output or update product recommendations, product application schedules, and the like based on this geographical data to best suit the local environment. The machine learning models 128 can include models such as decision trees, support vector machines, neural networks, and the like.

The visualization system 102 can use the machine learning models 128 or other software programs or modules to identify correlations between genetic markers and skin health. The machine learning models 128 use these correlations to predict how a user's skin may respond to different beauty products and treatments, or other software programs/modules can retrieve expected responses from a database or other data storage. The machine learning models 128 can output or update product recommendations, product application schedules, and the like based on the genetic information. Inputs can be additionally provided from known or detected family members and predictions made regarding likely effects on a user based on product effects on a family member. Predictions can include predictions of potential allergic or adverse reactions based on the user's genetic data or based on user knowledge of same or similar products to which the user has had an adverse reaction in the past. Outputs of the machine learning models 128 or other software programs or modules therefore can include adjustments to recommendations and personalized regimens based on problematic skin care ingredients.

In some examples, one or more machine learning model(s) 128 may be executed on the visualization system 102, while in other examples one or more machine learning model(s) 128 may be executed on another computing system, separate from the visualization system 102. For instance, the visualization system 102 may send data to another computing system, where a trained machine learning model 128 is applied to the data, and the other computing system may send a prediction or recommendation, based upon applying the trained machine learning model 128 to the data, to the visualization system 102. Moreover, in some examples, one or more machine learning model 128(s) may be trained by respective machine learning model training application(s) 130 executing on the visualization system 102, while in other examples, one or more machine learning model(s) 128 may be trained by respective machine learning model training application(s) executing on another computing system, separate from the visualization system 102.

Whether the machine learning model(s) 128 are trained on the visualization system 102 or elsewhere, the machine learning model(s) 128 may be trained by respective machine learning model training application(s) 130 using training data (including historical data in some cases), and the trained machine learning model(s) 128 may then be applied to new/current data that is separate from the training data in order to determine, e.g., predictions and/or identifications related to the new/current data.

For example, a machine learning model 128 trained to generate visualizations of different skin care regimens may be trained by a machine learning model training application 130 using training data including genetics of multiple (e.g., hundreds or thousands) of users or of an entire regional population, and images of those users. For example, products that were successfully used by a group of users having a particular genetic profile may have resulted in a particular change to the user's appearance, for example to the skin on their face or portion thereof. The machine learning model 128 can therefore be trained to learn how products affected user appearance, and the visualization system 102 or processor 116 can apply those effects to visualizations (e.g., images) by modifying images or visualizations to include or account for the predicted effects. For example, a cream found to reduce acne by 5%, 10%, etc. in a population, when applied to a visualized skin care regimen, may result in a visualization with 5%, 10% or other reduction in acne depending on a time duration over which the product was used. As another example, a user wishing to see the effect of using an acne treatment for a period of time can provide a user request and the visualization system 102 can respond with a visualization of how users with similar genetics were affected by using the acne treatment for the same period of time.

As another example, a machine learning model 128 trained to generate visualizations of skin age progression may be trained by a machine learning model training application 130 using training data including genetics, lifestyle, environment, current skin condition, current skin care regimens and other data of multiple users, in addition to images those users. Images can be labeled with user ages. The machine learning model 128 can therefore be trained to learn how a different user's skin will age given the user's current image, genetics, lifestyle, environment, current skin care regimen and current skin condition.

As another example, a machine learning model 128 trained to analyze data associated with a skin care regimen may be trained by a machine learning model training application 130 using training data including: genetics of multiple (e.g., hundreds or thousands) of users or of an entire regional population, geographical information, a history of products successfully used by that group of users, and other relevant inputs. For example, products that were successfully used by a group of users having a particular genetic profile may have resulted in positive changes to the users' skin health, either subjectively as reported by the users or as measured by skin care practitioners or devices. The machine learning model 128 can therefore be trained to learn which products or product types should be recommended for users of similar genetics. As another example, products that were successfully used by a group of users in a geographic location may have resulted in positive changes to the users' skin health, either subjectively as reported by the users or as measured by skin care practitioners or devices. The machine learning model 128 can therefore be trained to learn which products or product types should be recommended for users in that geographical region or regions of a similar climate.

As another example, a machine learning model 128 trained to generate a visualization of a skin care regimen may be trained by a machine learning model training application 130 using training data including images of multiple users. For instance, a personal care regimen for a person can be labeled with the particular products used, the ingredients/formulations of the products, any scheduling or timing of the regimen, etc., and these labeled regimens may be used as training data. The images can be labeled with regimens for each user and an indication or evaluation as to whether the skin care regimen was beneficial. Once sufficiently trained using this training data, such a machine learning model 128 may be applied to a new person, a new image of the same person or a different person, etc., such as an image provided by a user via a user interface 120, or an image from a social media, and the machine learning model 128 can identify or predict personal care products for the new person or based on the new image, that would be beneficial based on the learning. Effects of applying this skin care regimen can be learned during this same process and applied to the image provided by the user or to a stored image.

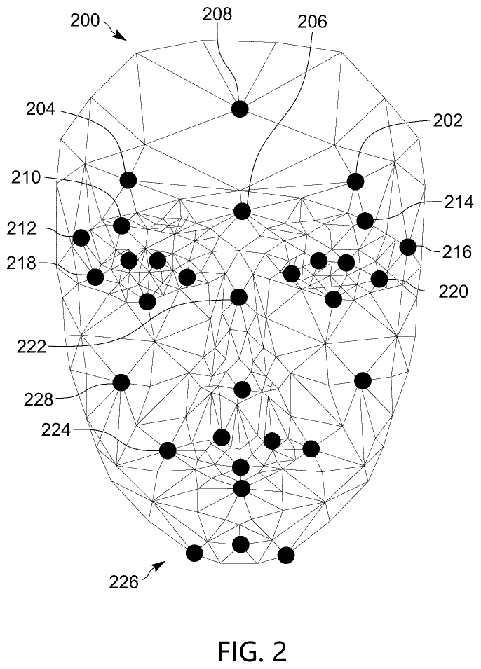

Moreover, as another example, a machine learning model 128 trained to provide visualizations of a care regimen can be trained by a machine learning model training application 130 using training data including images associated with various individuals' skin, and indications of skin types, skin health conditions, or other skin characteristics associated with the various individuals' skin. For instance, images of individuals having various skin types may be labeled with the respective skin types shown in each image. Similarly, images of individuals having various skin health conditions may be labeled with an indication of the health condition, the location of visual indicators associated with the health condition shown in the image, etc. Furthermore, images of individuals having various genetic traits may be labeled with the respective genetic traits. These labeled images may be used as training data, and once sufficiently trained using this training data, such a machine learning model 128 may be applied to a new image, video, and/or three-dimensional map associated with a user's face (e.g., a 3D map generated as described with respect to FIG. 2 later herein or as generated for display by the display device 106), and may identify/predict a skin type, skin health condition, genetic condition and/or other skin characteristic associated with the user's face. The skin type or health condition can be matched with products or formulations known to be beneficial to that skin type/condition/genetics, either as learned by the machine learning model 128 or as stored in lookup tables or other databases. The visualization system 102 can provide a personalized skin care regimen based on the learning, and a visualization can be updated based on the generated skin care regimen.

Additionally, as another example, a machine learning model 128 trained to generate skin care regimen visualizations may be trained by a machine learning model training application 130 using any updated training data based on user feedback, product formulation changes, new product availability, and the like. Recommendations can be updated by other types of software applications or modules based on scientific discoveries, changes in the user's skin as captured by the imaging system 112 or user device 104, location data or geographical changes pertaining to the user or similar users, etc. The machine learning model 128 may be trained by a machine learning model training application 130 using training data including products selected by previous users, characteristics of the previous users, input/feedback from the previous users about the products, etc. For instance, various products may be labeled with indications of characteristics of users who gave positive feedback regarding the products, indications of similar products receiving positive or negative feedback, etc. Once sufficiently trained using this training data, such a machine learning model 128 may be applied to a user, the user's characteristics, and previous care products selected/liked by the user and may predict/suggest other products that the user may enjoy or provide personalization suggestions. Visualizations may be updated or generated to incorporate execution or implementation of the updated skin care regimen.

In various aspects, the machine learning model(s) 128 may comprise machine learning programs or algorithms that may be trained by and/or employ neural networks, which may include deep learning neural networks, or combined learning modules or programs that learn in one or more features or feature datasets in particular area(s) of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques.

In some embodiments, the artificial intelligence and/or machine learning based algorithms used to train the machine learning model(s) 128 may comprise a library or package executed on the visualization system 102 (or other computing devices not shown in FIG. 1). For example, such libraries may include the TENSORFLOW based library, the PYTORCH library, and/or the SCIKIT-LEARN Python library.

Machine learning may involve identifying and recognizing patterns in existing data (such as training a model based upon historical data) to facilitate making predictions or identification for subsequent data (such as using the machine learning model on new/current data order to determine a prediction or identification related to the new/current data).

Machine learning model(s) may be created and trained based upon example data (e.g., “training data”) inputs or data (which may be termed “features” and “labels”) to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs for the model, executing on the server, computing device, or otherwise processor(s), to predict, based upon the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated. The disclosures herein may use one or both of such supervised or unsupervised machine learning techniques.

In addition, memories 118 may comprise a computer-readable medium or computer-readable media that may also store additional machine-readable or computer-readable instructions, including any of one or more application(s), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For instance, in some examples, the computer-readable instructions stored on the memory 118 may include instructions for carrying out any of the steps of the method 500 via an algorithm executing on the processors 116, which is described in greater detail below with respect to FIG. 5. It should be appreciated that one or more other applications may be envisioned and that are executed by the processor(s) 116. It should be appreciated that given the state of advancements of mobile computing devices, any or all of the processes functions and steps described herein may be present together on a mobile computing device, such as the user device 104, the imaging system 112 or the display device 106.

### 3D Face Model Creation Process

FIG. 2 illustrates a three-dimensional (3D) face model creation process according to some embodiments. The face model can be generated by the visualization system 102 described earlier herein. Once the face model is created, the effects of the skin care regimen can be applied to the model as described later below and the model can then be provided, in whole or in part and/or in a variety of views, to the display device 106 or the user device 104. This allows users to visualize the effects of each treatment on the user's skin, and to visualize effects of compliance or non-compliance with a recommended skin care regimen.

In some embodiments, the user device 104 or imaging system 112 may be configured to provide image data substantially in real-time to the visualization system 102, and the visualization system 102 may be configured to generate or manipulate the 3D face model 200 substantially in real-time from the provided image data. The visualization system 102 may transmit data indicating the 3D face model 200 back to the user device 104 or to the display device 106, which may use the received data to display or adjust a representation of the 3D face model substantially in real-time from the initial obtaining of image data at the user device 104 or imaging system 112.

The 3D face model 200 identifies each of a plurality of points on the face of the user and/or on surrounding body parts (e.g., the scalp, hair, neck, etc.). For example, points 202, 204, 206 can define a hairline, and point 208 can define a point within the hair or on the forehead. In some embodiments, each point lies at the intersection of two or more lines connecting the identified points. Each identified point may be associated with positional information (e.g., positions in the x-, y-, and z-axes), color information (e.g., hue, saturation, brightness, etc.), and/or other information. More particularly, the 3D face model 200 may identify each point as corresponding to a facial feature of the user or a particular portion thereof (e.g., tips or corners of eyebrows with points 210, 212, 214, 216; points 218, 220 along the eye or corners thereof that can define the eye points at corner of a mouth, a point 222 defining an edge of a nose, a point 224 defining a part of a lip, a point 226 defining the chin, a point 208 defining hair, eyelash, etc.). Not all points are labeled, to avoid clutter within the 3D face model 200 illustrated in FIG. 2.

In embodiments, generating the 3D face model 200 includes iteratively identifying and evaluating points (e.g., any of the points shown in FIG. 2) on the face (and/or surrounding parts) of the user to identify points corresponding to particular facial “landmarks” of interest (e.g., a point 224 defining one or more corners of the mouth, a point 222 defining the top of the nose, other feature of the nose, various points 218, 220 defining the eyes, points 202, 204, 206 defining the edge of the hairline, cheekbones 228, a point 226 defining the chin, etc.). For example, based upon information associated with facial points identified in a first iteration of generating the 3D face model 200, a second, third, fourth, etc. iteration(s) of generating the 3D face model 200 may be executed to iteratively identify points closer to the landmarks of interest until each point of interest is positively identified. The facial points may thus correspond to the particular facial landmarks of interest, as iteratively determined via these techniques. Identified features can include features such as lip, nose, ear, forehead, cheek, hairline, piercing, tattoo, wrinkle, pimple, mole, scratch, scar tissue, and the like.

Each of the identified features may have various identified characteristics associated therewith, including for example position, angular orientation, color, tone, condition of skin contained therein (e.g., oily, dry, smooth, wrinkled, stretched, etc.), relative arrangement to another identified feature(s), etc. Where multiple instances of any particular feature exist on the user's face (e.g., as is the case with eyes, ears, cheekbones, etc.), each instance of the feature is identified and considered independently, so as to account for the user's natural facial asymmetries and/or other variations among the user's facial features.

Generating the 3D face model 200 based upon obtained image data may include the use of various artificial intelligence (AI) and/or computer vision techniques. Particularly, AI and/or computer vision techniques for generating the 3D face model 200 may include machine learning and/or computer vision techniques, including but not limited to deep learning, artificial neural networks (fuzzy neural networks, feedforward neural networks, convolutional neural networks, etc.), hidden Markov models, classification, clustering, principal component analysis (PCA), discrete cosine transform (DCT), linear discriminant analysis (LDA), locality preserving projection (LPP), Gabor wavelet techniques, independent component analysis (ICA), generative adversarial networks (GANs), federated learning, and/or other approaches for facial identification/recognition/generation. It should be appreciated that generating the 3D face model 200 may comprise various new or existing techniques, particularly including new or existing AI techniques (e.g., new or existing machine learning techniques). These new or existing techniques may include open source techniques, proprietary techniques, and/or other techniques, including combinations thereof. As will be described further in subsequent sections, AI techniques such as those described above may additionally or alternatively be applied to other systems and methods of this disclosure, for example systems and methods for adapting the 3D face model 200, predicting changes to the 3D face model 200 based on application or use of a skin care regimen or portion thereof and based on various levels of compliance with the skin care regimen, recommending skin care products or routines, and the like.

The 3D face model 200 can be enhanced with information that accurately represents the user's current skin. For example, data can be retrieved from other images regarding wrinkles, blemishes, hyperpigmentation, and the like, and superimposed on the 3D face model 200. As will be described in further detail herein, techniques of this disclosure may include analyzing and/or manipulating identified features from the 3D face model 200 to, for example (1) provide a visualization of possible effects of application of skin care treatments, adherence to a schedule of application of skin care treatments, etc., (2) generate and provide recommendations of skin care products or routines for a given feature(s), and/or (3) verify whether any step of a skin care regimen routine was successfully completed. In examples, point (3) may include time lapse or time delayed information to account for an amount of time that a skin care regimen is followed or since the beginning of implementation of the skin care regimen.

Use of the technologies of this disclosure may include repeatedly or continuously regenerating and/or adjusting the 3D face model 200 based upon new image data obtained via the user device 104, imaging system 112 and/or via other sources. Accordingly, feature identification with respect to the 3D face model 200 may include updating and tracking the respective positions of features, e.g., as newly obtained image data reflects the user repositioning, rotating, and/or changing their facial expression while within the frame of a device camera while capturing images. The 3D face model 200 can be displayed in an AR environment, enabling the user to interact with the model and inspect it from various angles to observe the mapping of different anti-aging treatments onto the model. The interface can also include controls for toggling between different treatment scenarios.

In a further embodiment, the system 100 can map a wide range of anti-aging treatments onto the 3D face model 200. These treatments can be stored in a comprehensive database, each associated with specific effects and outcomes based on scientific research and clinical studies. The system 100 can use machine learning algorithms to match the potential outcomes of each treatment with the user's specific situation (e.g., genetics, geographic location, time of year and the like), creating a visualization of the potential effects of each treatment on the user's skin.

### Example User Interface Displays

FIGS. 3A-3C depict exemplary user interface displays as may be provided by a user interface for a user of the system 100 (e.g., a user interface 120 of the user device 104). In some embodiments, certain displays or depictions of the personal care regimen, before/after images, 3D images, etc. can be provided in a separate device such as another user device similar to the user device 104 (e.g., a second smartphone or tablet, desktop computer, laptop, etc.) display device 106 and the like.

FIG. 3A illustrates an example user interface display via which a user can provide information to the system 100. The information can be used by the visualization system 102 in generating 3D face model 200, for providing skin care regimens and other recommendations, and the like. The user can provide identifying data (e.g., name, age, nicknames, address, and the like) in text boxes 300, 302 or other similar input mechanisms. A list 304 of conditions can be provided, which can include conditions identified by the visualization system 102 or associated measurement devices, imaging system 112, cameras associated with the user device 104, and the like. The list 306 can include any possible or detected habits, in particular habits that may affect skin care and skin health.

Other user interface items can include buttons 308, 310, 312 that can open other dialog boxes, interactive dialog systems, or interactive displays for obtaining further data from the user. For example, buttons 308, 310, 312 or similar interface items can allow a user to upload genetic data, import data from a database, upload medical history, add further conditions or habits, and the like. Other user interface elements and mechanisms can include questionnaires, interactive dialogue systems, and options to import data from relevant external sources or databases.

FIG. 3B illustrates details of the recommended skin care regimen. List 314 includes a list of one or more products currently within the recommended skin care regimen. The list 314 can include brand names or categories and can the visualization system 102 or other component of system 102 can update the list 314 as the skin care regimen is updated. The display can include details on a schedule 316 for using a selected product 318. The schedule 316 can include dates, intervals, time of day, and other information. Various other interface items 320, 322 can be included. For example, a user can adjust products using interface item 320 or a schedule using interface item 322.

The system 100 can automatically populate the list 314 with recommended products. In addition, the user can manually add products to the list 314. The products in the list 314 can be automatically updated by the system 100 when machine learning algorithms or other components of the system 100 determine that products have changed, or that the recommendation is to be changed, or that the product list is to be changed. The interface item 320 allows users to manually change items in the regimen. Users could also adjust or add details about the products in the list 314, including dates purchased, where bought, etc. Similarly, a user can adjust schedules for product use with interface item 322.

FIG. 3C illustrates an example graphic 324 of the user or similar user's skin. The graphic 324 can include views of the user's skin if the user were not using the program. The graphic 324 can also include views of the user's skin with or without use of individual skin care products. For example, the user could request a visualization of how his or her skin would change with application of an individual moisturizer or exfoliant. The visualization system 102 can generate the graphic 324 according to methods described above with reference to FIG. 2. The graphic 324 can be the same or similar to the 3D face model 200 described earlier herein, and/or the graphic 324 can represent the face or a facial feature of the user or of a user having similar genetics, skin tone, etc. The user can rotate the graphic 324 using elements 326. The user can visualize the effects of omitting, terminating or adding certain skin care operations/treatments using elements 328, 330 (e.g., reversion elements) and the user can revert to different visualizations using elements 328, 330. The user can tilt or shift the graphic 324 using elements 332, 334, and/or 336. The visualization system 102 can use photographic aging software or predictions based on increased or decreased product use, a worsening or improvement in certain skin conditions, and the like to update the graphic 324. In addition, or alternatively, the visualization system 102 can access facial aging models of persons having similar genetics or persons in a similar ethnic group as the user to generate predictive images.

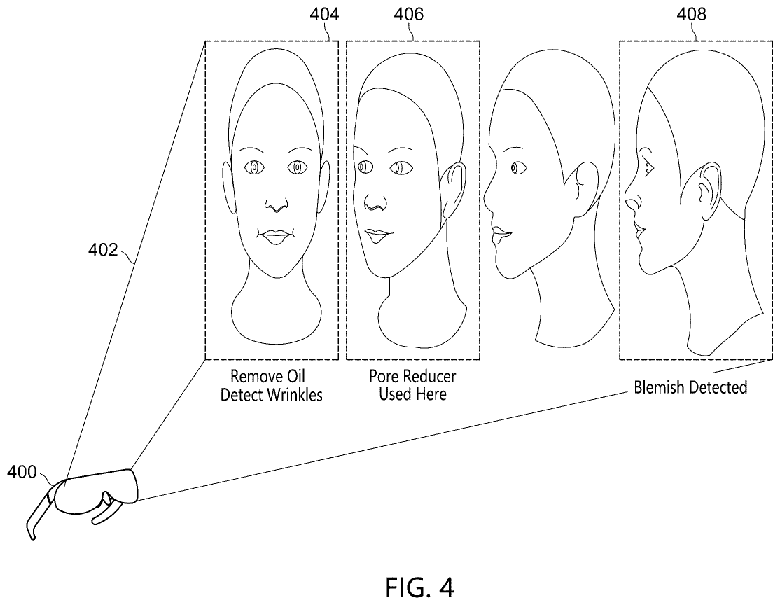

A similar graphic can be displayed on other types of systems. For example, FIG. 4 depicts an example visualization that may be provided by a user interface associated with a system according to some embodiments. In the example, AR goggles 400 or a similar device can display an interface 402. The interface 402 can provide various graphics that may be similar to graphics (e.g., 3D face model 200) developed according to methods described with reference to FIG. 2. The user can interact with their 3D face model in the AR interface. Users can inspect the projected outcomes of recommended treatments, visualize the potential effects of non-adherence to treatments, compare different treatment scenarios side-by-side (e.g., in a simultaneous fashion), and observe the potential future skin condition under various lifestyle and environmental conditions. The AR interface 402 or overlays 404, 406 408 can provide access to scientific information and education resources related to benefits of the skin care regimen.

The visualization provided in the interface 402 can include a time-lapse feature to illustrate potential outcomes that reflect compliance and non-compliance with the skin care regimen. The time-lapse feature may display results illustrating a short-term outcome and a long-term outcome. The time-lapse feature may illustrate potential outcomes resulting from one or more percentages of compliance with the skin care regimen. The time-lapse feature can use images of users of similar ethnic or genetic groups at various life stages, and/or the time-lapse feature can use images of the user being visualized. The time-lapse feature can extrapolate features of the user as the features could appear under various treatment scenarios. For example, features of the user could be depicted according to a current acne status, and extrapolated to remove or add various acne features such as pimples, whiteheads, blackheads, etc., varying according to predicted treatment outcomes predicted by trained machine learning models.

Different overlays 404, 406, 408 can be provided and the user can interact with the overlay to see effects of different treatments, request or be alerted of detections made in changes to skin conditions, and the like. For example in overlay 404, a user could interact to view the interface 402 with oiliness removed from the user's face, or be notified that a wrinkle weas detected below the chin. Another overlay 406 (or the same overlay) could be used to view what products were used in a particular portion of the face for example. Another overlay 408 could provide focus or alerts regarding blemishes. In some embodiments, alerts can be accompanied by notification or advice of an action to be taken regarding the skin care regimen. For example, the user can be alerted to apply a product, or to avoid applying a particular product. The notification could be provided to the system 100 or advice could be provided to the user, or the like. It will be appreciated that while FIG. 4 provides a few examples of features that could be provided by the system 100, the AR goggles 400 and interface 402 can provide any number of overlays, view types, advice, interaction, and the like to allow the user to visualize a skin care regimen and all facets thereof.

### Example Method

FIG. 5 depicts a flow diagram of an exemplary computer-implemented method for providing visualization of results of application of a skin care product or implementation of a skin care regimen including one or more skin care products, according to one embodiment. One or more operations of the method 500 may be implemented as a set of instructions stored on a computer-readable medium or memory (e.g., memory 118, memory 126, etc.) and executable on one or more processors (e.g., processor 116, processor 124, etc.).

The method 500 may begin with operation 502 with obtaining training data including skin characteristics for a population of users, an indication of the respective skin care products used on the population of users, and a respective treatment outcome for each user. In some examples, operation 502 can include collecting information such as demographic information, medical history, genetic data, lifestyle data, historical skin data, user diet, sleep patterns, exercise habits, tobacco use, alcohol use, geographic location, information regarding the geographic location including weather or environmental pollution concentrations, and the like. In examples, this can be done through a user interface such as shown in FIG. 3A. In some examples, collecting can be done by receiving data or analysis related to a deoxyribonucleic acid (DNA) sample of the user. For example, data or analysis can be retrieved from or provided by a genetic testing service, ancestry research organization/service, and the like. In still other examples, collecting includes accessing a genetic testing service (e.g., by the user accessing a service website, or with permission of the user, etc.).

The method 500 can further include generating a skin care regimen using one or more skin care products. The regimen can be generated based on analysis of images, for example images used in generation of the 3D face model 200. Images can include still images, a video or video frames, thermal images, and the like. The visualization system 102 can adjust the skin care regimen (or provide notification/recommendation to a manufacturer of a product in the skin care regimen) based on a determination that a number of users exhibit less than perfect compliance with the skin care regimen. For example, if less than perfect compliance is detected, the product could be reformulated at a higher strength (or otherwise reformulated) where feasible/safe so that effects are detectable even with less than perfect compliance with the skin care regimen. Less than perfect compliance can be detected by receiving user input that a user is not complying with the skin care regimen, analyzing social media posts to detect dissatisfaction or lack of compliance with the skin care regimen, and the like.

In some examples, the method 500 and/or generation of the skin care regimen can include using a trained machine learning model that is trained using genetic profiles of a population to generate a skin care regimen that would benefit members of the population. The model can be updated using expanded training data of a second population that is a superset of the initial population. For example, once a machine learning model is trained using an initial geographic population or group of users having same or similar genetics, the machine learning model can be trained again or updated using a larger geographic population or group that may include at least the initial geographic population or group of users. The model can also be updated or trained to predict personal care conditions that are prevalent among one or more of an ethnic group, a cultural group, or a national group.

The method 500 can continue with operation 504 with training a machine learning model, using the training data, to predict a treatment outcome for a new user based on skin characteristics of the new user and on an indication of the skin care product used on the new user. Operation 504 can be done using a trained machine learning model (e.g., as described above with reference to elements 128, 130), although some operations can additionally or alternatively be implemented in other types of software applications or modules. In some examples, operation 506 can be executed using reinforcement learning to generale improved predictions of effects of products to be applied in the skin care regimen. Operation 506 can include comparing expected skin improvement with actual skin improvement and updating treatment-result matching based on this comparison.

Updates to the trained machine learning model 128 can be based on feedback input by the user, among other modes, inputs or methods of updating. Feedback can include commentary on the visualization provided by the visualization system 102, reviews of products, comments on products or properties thereof, statements regarding whether products had desired effects on the user's skin, and the like. The feedback can be provided in the form of end-user actions to retrain and improve the corresponding machine learning model by helping the machine learning model determine whether previous learning was in error. The feedback could also be used to adjust other software applications or modules that analyze skin care regimens, or that provide visualizations, or any other type of software application/module used herein.

In addition to product formulation information, other data or types of data can serve as inputs to machine learning models or other software applications and modules. This other data can include age, gender, ethnicity, skin characteristics, historical skin data, lifestyle data (e.g., tobacco and alcohol use, time spent outdoors) or genetic factors of the user. For example, machine learning models can be trained to provide visualizations of skin care regimens, or recommendations for a skin care regimen, based on learned knowledge of users having similar skin characteristics, lifestyles/lifestyle choices, or genetic characteristics. Similarly, still further inputs can be considered including diet, sleep patterns, exercise, geographic location, local weather and climate, local environmental pollutant concentrations, and the like. User device 104 location information can be accessed to detect if a permanent or temporary change to the skin care regimen should be made. For example, if the user lives in the Northeast United States and travels to Florida for a month (e.g., is in Florida only temporarily), presence in Florida can cause the skin care regimen to be temporarily adjusted to account for increased sunlight, heat and humidity. In other embodiments, the system 100 can refrain from updating skin care recommendations or make only limited recommendations if the user location is expected to be only temporary.

The method 500 can continue with operation 506 with generating a visualization of treatment outcomes. The visualization may include a 3D face model (e.g., similar to 3D face model 200 (FIG. 2) of a user face or a user facial feature. The visualization can be provided on an AR device (e.g., display device 106 (FIG. 1) or AR goggles 400 (FIG. 4)). A user interface (e.g., as described with reference to FIG. 3A-3C) can allow users to enable or display a different visualization reflecting a different skin care regimen, to tilt or rotate the visualization, to focus in on different aspects of the 3D face model, and the like.

In some example embodiments, a baseline analysis of a model can be provided, and this baseline can be visualized, stored, or the like. The baseline can include a visualization of skin age progression based on at least one of currently-used skin care products, current skin condition, genetics, lifestyle, or environmental factors.

The method 500 can continue with operation 508 with providing the visualization to a display. The display can include any or all components and features described above with reference to FIG. 2-4.

Other features can be provided in the system 100 and implemented using the method 500. For example, in some embodiments, the method 500 can include encrypting at least one of the visualization and the skin care regimen. Still further, the visualization system 102 or user device 104 can include or be provided with an interface to a third-party consultant. The user can receive advice from the third-party consultant. In some examples, the advice can be based on or be in response to the user or component of the system 100 providing the visualization or the skin care regimen to the third-party consultant. Similarly, interfaces can be provided to retailers (online or otherwise), to facilitate purchasing products recommended in the skin care regimen.

The method 500 can include providing estimates of financial implications of complying with the skin care regimen (e.g., product costs) alongside or proximate other estimates of healthcare costs associated with not complying with the skin care regimen. For example, costs of skin cancer treatments can be compared to costs of sunscreen products, to provide further motivation/education to the user. The system 100 can be tied into gamification programs to provide additional incentives. For example, gamification can improve user engagement and increase compliance with the skin care regimen by creating a game to accomplish a skin care goal, personal care goal or other health goal of the user. Inputs to the game can include user mood, user usage of products, or other performance measurements.

### ADDITIONAL CONSIDERATIONS

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” or “some embodiments” means that a particular element, feature, structure, or characteristic described in connection with the embodiment is included in at least one embodiment. The appearances of the phrase “in one embodiment” or “in some embodiments” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of “a” or “an” is employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system for visualizing a skin care regimen and/or systems, methods, and/or techniques associated therewith. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

### ASPECTS

1. A computer-readable medium including instructions that, when executed on a processor, cause the processor to perform operations for providing a visualization of results of application of a skin care product, the operations including: obtaining training data including skin characteristics for a population of users, an indication of the respective skin care products used on the population of users, and a respective treatment outcome for each user; training a machine learning model, using the training data, to predict a treatment outcome for a new user based on skin characteristics of the new user and on an indication of the skin care product used on the new user; generating a visualization of the treatment outcome based on applying the trained machine learning model to an image of the new user; and providing the visualization to a display.

2. The computer-readable medium of claim 1, wherein: the training data further includes at least one of historical skin data, lifestyle data, or genetic factors and corresponding treatment outcomes for each user the population of users, and the machine learning model is trained to predict the treatment outcome for the new user further based on at least one of historical skin data, lifestyle data, or genetic factors of the user.

3. The computer-readable medium of claim 1, wherein: the training data further includes at least one of geographic location, weather conditions, and local environmental pollutant concentrations and corresponding treatment outcomes for each user of the population of users; and wherein the machine learning model is trained to predict the treatment outcome for the new user further based on at least one of geographic location, weather conditions, and local environmental pollutant concentrations for the new user.

4. The computer-readable medium of claim 3, wherein the operations further comprise: detecting change in user geographic location to a new geographic location; and wherein the machine learning model is trained to predict the treatment outcome for the new user further based on the new geographic location.

5. The computer-readable medium of claim 4, wherein the operations further comprise: detecting whether the user is permanently or temporarily in the new geographic location; and refraining from training the machine learning model to predict the treatment outcome for the new user based on the new geographic location if the user is temporarily in the new geographic location.

6. The computer-readable medium of claim 1, wherein the training data further includes at least one of user age, user gender, or user ethnicity, and corresponding treatment outcomes for each user of the population of users, and wherein the machine learning model is trained, using the training data, to predict the treatment outcome for the new user based on at least one of user age, user gender, and user ethnicity.

7. The computer-readable medium of claim 1, where the operations further include: providing a skin care regimen for the new user, based on predicted treatment outcomes for the new user of using indicated skin care products.

8. The computer-readable medium of claim 7, wherein the operations further include comparing predicted treatment outcomes with actual treatment outcomes obtained from images of the user and providing feedback to the trained machine learning model based on the comparing.

9. The computer-readable medium of claim 7, wherein the operations further include: accessing at least one of product formulation updates or product availability updates from a product database, and wherein the training data further includes at least one of product formulation updates or product availability updates; and wherein the machine learning model is trained to predict the treatment outcome for the new user further based on at least one of product formulation updates or product availability updates.

10. The computer-readable medium of claim 7, wherein the training data further includes an indication of the skin care regimen used on the population of users and corresponding treatment outcomes the operations further comprise: training the machine learning model, using the training data, to predict a treatment outcome for the new user, based on skin characteristics of the new user and on an indication of the skin care regimen used by the new user; and generating a visualization of the treatment outcome.

11. The computer-readable medium of claim 7, wherein the operations further comprise providing a notification to a product manufacturer to adjust formulation of a skin care product upon receiving user input that at least one user is exhibiting less than perfect compliance with the skin care regimen.

12. The computer-readable medium of claim 7, wherein providing the visualization comprises providing a time-lapse feature to illustrate potential outcomes that reflect compliance and non-compliance with the skin care regimen by (i) providing a first image depicting a short-term outcome of complying with the skin care regimen and a second image depicting a short-term outcome of not complying with the skin care regimen, and (ii) providing at least a third image depicting a long-term outcome of complying with the skin care regimen and at least a fourth image depicting a long-term outcome of not complying with the skin care regimen, and (iii) providing at least the first image, second image, third image and fourth image to a display, wherein the at least first image, second image third image and fourth image are generated based on extrapolation of features of the user according to predicted treatment outcomes.

13. The computer-readable medium of claim 7, wherein providing the visualization comprises receiving user input to enable or display a different visualization, the different visualization reflecting use of a different skin care regimen, and wherein the operations comprise providing a simultaneous comparison of the visualization and the different visualization.

14. The computer-readable medium of claim 1, wherein the operations further comprise updating the machine learning model based on user feedback pertaining to perceived accuracy of the machine learning model.

15. The computer-readable medium of claim 1, wherein the operations further comprise updating the machine learning model based on user feedback pertaining to user satisfaction with the visualization.

16. The computer-readable medium of claim 1, wherein the machine learning model is trained using a reinforcement learning algorithm.

17. The computer-readable medium of claim 1, wherein: the training data further includes images of the population of users and an indication of skin age progression, and at least one of currently-used skin care products, current skin condition, genetics, lifestyle, or environmental factors; and the operations further include training the machine learning model, using the training data, to predict skin age progression of the new user based on an image of the new user and at least one of the currently-used skin care products, current skin condition, genetics, lifestyle, or environmental factors for the new user.

18. The computer-readable medium of claim 1, wherein the image comprises a video or a frame of a video.

19. The computer-readable medium of claim 1, wherein the visualization includes a three-dimensional model of a user face or a user facial feature.

20. The computer-readable medium of claim 1, wherein providing the visualization comprises: providing the visualization on an augmented reality (AR) device.

21. The computer-readable medium of claim 1, wherein providing the visualization comprises providing a reversion feature to visualize effects of reversing or terminating a skin care action.

22. A method of providing a skin care augmented reality visualization, the method comprising: accessing an image of a user; analyzing skin characteristics of the user based on the image; providing a skin care regimen based on the skin characteristics; generating a visualization of the image as the image would appear, after a time lapse, with implementation of the skin care regimen; and providing the visualization to a display.

23. A system for providing a skin care augmented reality visualization, the system comprising: an image system configured to provide an image of a user; a display for displaying the image; and one or more processors coupled to the image system and to the display, the one or more processors configured to: analyze skin characteristics of the user based on the image; train a machine learning model to generate a skin care regimen based on the skin characteristics and on product information for skin care products; generate a visualization of the image as the image would appear, after a time lapse, with implementation of the skin care regimen; and provide the visualization to the display.

What is claimed is:

1. A computer-readable medium including instructions that, when executed on a processor, cause the processor to perform operations for providing a visualization of results of application of a skin care product, the operations including:
obtaining training data including skin characteristics for a population of users, an indication of the respective skin care products used on the population of users, and a respective treatment outcome for each user;
training a machine learning model, using the training data, to predict a treatment outcome for a new user based on skin characteristics of the new user and on an indication of the skin care product used on the new user;
generating a visualization of the treatment outcome based on applying the trained machine learning model to an image of the new user; and
providing the visualization to a display.

2. The computer-readable medium of claim 1, wherein:
the training data further includes at least one of historical skin data, lifestyle data, or genetic factors and corresponding treatment outcomes for each user the population of users, and
the machine learning model is trained to predict the treatment outcome for the new user further based on at least one of historical skin data, lifestyle data, or genetic factors of the user.

3. The computer-readable medium of claim 1, wherein:
the training data further includes at least one of geographic location, weather conditions, and local environmental pollutant concentrations and corresponding treatment outcomes for each user of the population of users; and
wherein the machine learning model is trained to predict the treatment outcome for the new user further based on at least one of geographic location, weather conditions, and local environmental pollutant concentrations for the new user.

4. The computer-readable medium of claim 3, wherein the operations further comprise:
detecting change in user geographic location to a new geographic location; and
wherein the machine learning model is trained to predict the treatment outcome for the new user further based on the new geographic location.

5. The computer-readable medium of claim 4, wherein the operations further comprise:
detecting whether the user is permanently or temporarily in the new geographic location; and
refraining from training the machine learning model to predict the treatment outcome for the new user based on the new geographic location if the user is temporarily in the new geographic location.

6. The computer-readable medium of claim 1, wherein the training data further includes at least one of user age, user gender, or user ethnicity, and corresponding treatment outcomes for each user of the population of users, and
wherein the machine learning model is trained, using the training data, to predict the treatment outcome for the new user based on at least one of user age, user gender, and user ethnicity.

7. The computer-readable medium of claim 1, where the operations further include:
providing a skin care regimen for the new user, based on predicted treatment outcomes for the new user of using indicated skin care products.

8. The computer-readable medium of claim 7, wherein the operations further include comparing predicted treatment outcomes with actual treatment outcomes obtained from images of the user and providing feedback to the trained machine learning model based on the comparing.

9. The computer-readable medium of claim 7, wherein the operations further include:
accessing at least one of product formulation updates or product availability updates from a product database, and wherein the training data further includes at least one of product formulation updates or product availability updates; and
wherein the machine learning model is trained to predict the treatment outcome for the new user further based on at least one of product formulation updates or product availability updates.

10. The computer-readable medium of claim 7, wherein the training data further includes an indication of the skin care regimen used on the population of users and corresponding treatment outcomes the operations further comprise:
training the machine learning model, using the training data, to predict a treatment outcome for the new user, based on skin characteristics of the new user and on an indication of the skin care regimen used by the new user; and
generating a visualization of the treatment outcome.

11. The computer-readable medium of claim 7, wherein the operations further comprise providing a notification to a product manufacturer to adjust formulation of a skin care product upon receiving user input that at least one user is exhibiting less than perfect compliance with the skin care regimen.

12. The computer-readable medium of claim 7, wherein providing the visualization comprises providing a time-lapse feature to illustrate potential outcomes that reflect compliance and non-compliance with the skin care regimen by (i) providing a first image depicting a short-term outcome of complying with the skin care regimen and a second image depicting a short-term outcome of not complying with the skin care regimen, and (ii) providing at least a third image depicting a long-term outcome of complying with the skin care regimen and at least a fourth image depicting a long-term outcome of not complying with the skin care regimen, and (iii) providing at least the first image, second image, third image and fourth image to a display, wherein the at least first image, second image third image and fourth image are generated based on extrapolation of features of the user according to predicted treatment outcomes.

13. The computer-readable medium of claim 7, wherein providing the visualization comprises receiving user input to enable or display a different visualization, the different visualization reflecting use of a different skin care regimen, and wherein the operations comprise providing a simultaneous comparison of the visualization and the different visualization.

14. The computer-readable medium of claim 1, wherein the operations further comprise updating the machine learning model based on user feedback pertaining to perceived accuracy of the machine learning model.

15. The computer-readable medium of claim 1, wherein the operations further comprise updating the machine learning model based on user feedback pertaining to user satisfaction with the visualization.

16. The computer-readable medium of claim 1, wherein the machine learning model is trained using a reinforcement learning algorithm.

17. The computer-readable medium of claim 1, wherein:
the training data further includes images of the population of users and an indication of skin age progression, and at least one of currently-used skin care products, current skin condition, genetics, lifestyle, or environmental factors; and
the operations further include training the machine learning model, using the training data, to predict skin age progression of the new user based on an image of the new user and at least one of the currently-used skin care products, current skin condition, genetics, lifestyle, or environmental factors for the new user.

18. The computer-readable medium of claim 1, wherein the image comprises a video or a frame of a video.

19. The computer-readable medium of claim 1, wherein the visualization includes a three-dimensional model of a user face or a user facial feature.

20. The computer-readable medium of claim 1, wherein providing the visualization comprises:
providing the visualization on an augmented reality (AR) device.

21. The computer-readable medium of claim 1, wherein providing the visualization comprises providing a reversion feature to visualize effects of reversing or terminating a skin care action.

22. A method of providing a skin care augmented reality visualization, the method comprising:
accessing an image of a user;
analyzing skin characteristics of the user based on the image;
providing a skin care regimen based on the skin characteristics;
generating a visualization of the image as the image would appear, after a time lapse, with implementation of the skin care regimen; and
providing the visualization to a display.

23. A system for providing a skin care augmented reality visualization, the system comprising:
an image system configured to provide an image of a user;
a display for displaying the image; and
one or more processors coupled to the image system and to the display, the one or more processors configured to:
analyze skin characteristics of the user based on the image;
train a machine learning model to generate a skin care regimen based on the skin characteristics and on product information for skin care products;
generate a visualization of the image as the image would appear, after a time lapse, with implementation of the skin care regimen; and
provide the visualization to the display.
